(19) United States
(12) Patent Application Publication
(10) Pub. No.: US 2023/0241262 A1
(43) Pub. Date: Aug. 3, 2023

(54) Wearable Shield 222, Covid & Other

(71) Applicant: Greg H. Louie, Calgary (CA)

(72) Inventor: Greg H. Louie, Calgary (CA)

(21) Appl. No.: 17/588,308

(22) Filed: Jan. 30, 2022

(51) Int. Cl. A61L 2/10

(52) U.S. Cl. CPC A61L 2/10

(57) ABSTRACT

The invention consists of utilizing UV Light (approximately 222 nm, but not limited to as technology improves) to be wearable to provide protection to the individual (PPE, personal protection equipment). This could entail any wearable technology to protect the wearer (person or animal) from bacteria that is potentially harmful. Wearable PPE for COVID or other variants or bacteria utilizing UV or other technology as it develops. This is also to cover wearable detection of harmful air born bacteria contaminants as technology develops in gas chromatography.

### FIELD AND BACKGROUND OF THE INVENTION

UV (ultraviolet) light technology is proven to be effective in reducing pathogens. UV 254 NM (nanometers) has been used commercially and effectively for various applications more commonly in water and HVAC systems. The UV 254 NM is known to be harmful to humans if exposed.

Recent studies have shown that UV (ultraviolet) light technology is effective in reducing pathogens particularly UV 222 NM has shown it is not harmful to humans when exposed but “effectively kills Sars-Cov-2”, “the virus that causes COVID-19”, according to study conducted by Hiroshima University (published in the American Journal of Infection Control).

The COVID-19 global pandemic has provided a need for personal protection equipment which has initially been in short supply. This pandemic has spawned the need for PPE (personal protective equipment) in the medical and general population. The PPE, such as face masks, currently available does not effectively filter the smaller airborne particles of the pathogen nor protect other facial areas that may be subject to exposure and subsequent contamination of the individual.

There are currently vaccines being developed at the time of writing which may or may not be effective against mutations currently being discovered.

There is a need for PPE that will protect a person from the various mutations and airborne particles that current masks to not protect against. This will be beneficial for those unable to take vaccines as well young people, infants, children etc. This also could help domestic animals including but not limited to household pets, cats, dogs, etc.

The background information is provided for the purpose of making known information believed by the applicant to be of possible relevance to the present invention. No admission is necessarily intended, nor should be construed, that any of the preceding information constitutes prior art against the present invention.

### SUMMARY OF INVENTION

In accordance with the present invention, the attachment of UV 222 NM apparatus to person or clothing is such a way as to provide a shield of protection to the individual against airborne pathogens such as COVID-19 or variants, etc. This will provide effective protection for the individual by killing most if not all the pathogens. The UV light shield will not harm the individuals nor hinder breathing or vision thus providing an advantage over conventional PPE (personal protective equipment). This can also be extended to sports equipment or animals (pets or domesticated animals) as required. This can also serve to sanitize hands or small devices such as cell phones as required.

### BRIEF DESCRIPTION OF FIGURES

The present disclosure will be better understood when read in conjunction with the following.

FIG. 1 illustrates the light apparatus attached to a hat providing a shield to protect against airborne Covid-19 or other bacteria pathogens.

FIG. 2 illustrates the light apparatus attached to vest or shirt irradiating a shield to protect against airborne Covid-19 or other bacteria pathogens.

FIG. 3 illustrates the light apparatus attached to a pole strapped to the person irradiating a cone shield to protect against airborne Covid-19 or other bacteria pathogens.

FIG. 4 illustrates the light apparatus attached to glasses and head lamp

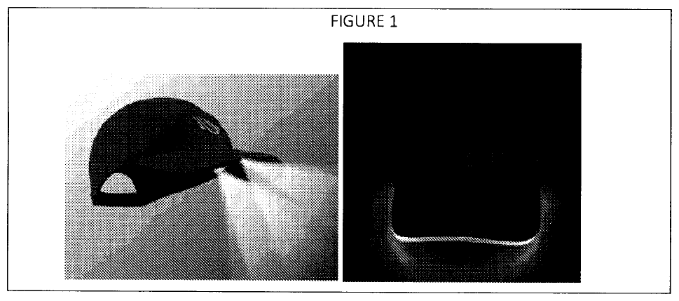

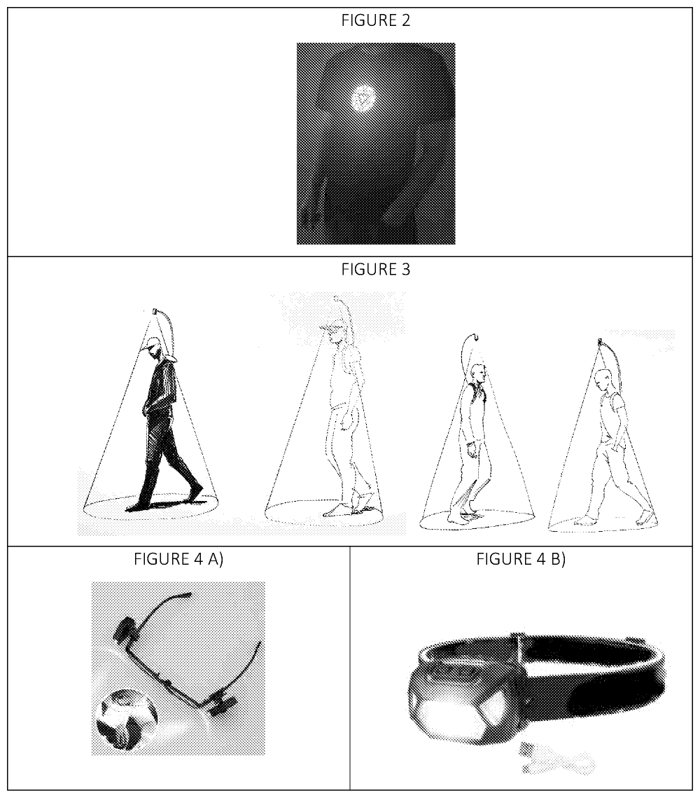

1. The Wearable Light Shield will provide better protection than currently available PPE against COVID-19 and other airborne pathogens utilizing the UVC 222 nm technology differently for the individual will provide better protection when outdoors where the existing fixed lighting apparatus will be absent.
The Wearable Light Shield of UV 222 nm will also provide means to sanitize hands and small personal items without physically cleaning with the current means of hand sanitizers of fluid type.
The Wearable Light Shield will also alleviate people with allergic reactions to some of the masks available.
The Wearable Light Shield will allow unmuffled speech while protecting against suspended airborne pathogens.
The Wearable Light Shield will allow people with breathing problems to be protected while not wearing masks. This will apply to sports activities as well where heavy breathing is encountered during activities.
